(19) United States
(12) Patent Application Publication
(10) Pub. No.: US 2023/0174940 A1
(43) Pub. Date: Jun. 8, 2023

(54) OVERCOMING IMMUNE SUPPRESSION WITH TGF-B RESISTANT NK CELLS

(71) Applicants: Dean Anthony LEE, Canal Winchester, OH (US); Prashant TRIKHA, Columbus, OH (US); Aarohi THAKKAR, Columbus, OH (US); Jennifer FOLTZ, St. Louis, MO (US); Meisam N. KARAROUDI, Columbus, OH (US); Jena E. MOSEMAN, Columbus, OH (US); RESEARCH INSTITUTE AT NATIONWIDE CHILDREN'S HOSPITAL, Columbus, OH (US)

(72) Inventors: Dean Anthony LEE, Winchester, OH (US); Prashant TRIKHA, Columbus, OH (US); Aarohi THAKKAR, Columbus, OH (US); Jennifer FOLTZ, St. Louis, MO (US); Meisam N. KARAROUDI, Columbus, OH (US); Jena E. MOSEMAN, Columbus, OH (US)

(21) Appl. No.: 17/922,248

(22) PCT Filed: Apr. 30, 2021
(86) PCT No.: PCT/US21/30142

§ 371 Date: Oct. 28, 2022

Related U.S. Application Data
(60) Provisional application No. 63/018,108, filed on Apr. 30, 2020.

(51) Int. Cl. C12N 5/0783; A61K 35/17; C07K 14/495; C07K 14/54; C07K 14/705

(52) U.S. Cl. CPC C12N 5/0646; A61K 35/17; C07K 14/495; C07K 14/54; C07K 14/70578; C12N 2501/15; C12N 2501/2321; C12N 2501/599; C12N 2502/99

(57) ABSTRACT

Disclosed are engineered feeder cells comprising soluble or membrane bound TGF-b and methods of their use in the production of NK cells resistant to TGF-b and use of said generated TGF-b resistant NK cells to treat a cancer.

This Application claims the benefit of U.S. Provisional Application No. 63/018,108, filed on Apr. 30, 2020, which is incorporated herein by reference in its entirety.

### I. BACKGROUND

1. The use of NK cells in anti-cancer therapy is increasing. However, studies have shown that cancers secrete TGF-β that can reduce the killing action of NK cells. Previous efforts to address the decreased killing have included the use of TGF-β inhibitors; however, the use of TGF-β inhibitors can cause adverse side effects systemically. What are needed are new reagents and methods that can be used to preserve the killing action of NK cells in the presence of TGF-β (i.e., TGF-β resistant NK cells).

### II. SUMMARY

2. Disclosed are methods and compositions related to engineered feeder cells and use of said feeder cells to generated NK cells resistant the TGF-β.

3. In one aspect, disclosed herein are engineered feeder cells that has been modified to express soluble or membrane bound TGF-β. For example, the TGF-β (either soluble or membrane bound) can be operatively linked to a constitutive or inducible promoter including, but not limited to, a CMV or EF1A promoter.

4. Also disclosed herein are engineered feeder cells of any preceding aspect, further comprising at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL.

5. In one aspect, disclosed herein are engineered feeder cells of any preceding aspect, wherein the feeder cells comprise PBMCs, RPMI8866, HFWT, K562 cells, EBV-LCL, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-21, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound 4-1BBL, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-15 and 4-1BBL , or NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-21 and 4-1BBL.

6. Also disclosed herein are plasma membrane particles or exosomes derived from the engineered feeder cell of any preceding aspect. In some instances the particles or exosome can be obtained by nitrogen cavitation.

7. In one aspect, disclosed herein are methods of generating TGF-β resistant NK cells, comprising incubating NK cells in the presence of the engineered feeder cells, plasma membrane particles, or exosome of any preceding claim. For example, disclosed herein are methods of generating TGF-β resistant NK cells, comprising incubating NK cells in the presence of feeder cells (including but not limited to PBMCs, RPMI8866, NK-92, NK-92MI, NK-YTS, NK, NKL, ML, ML C.2, NK 3.3, NK-YS, HFWT, K562 cells, EBV-LCL, NK cells transfected with membrane bound IL-21, NK cells transfected with membrane bound 4-1BBL, NK cells transfected with membrane bound IL-15 and 4-1BBL , or NK cells transfected with membrane bound IL-21 and 4-1BBL) that have been engineered to express TGF-β or incubating NK cells in the presence of plasma membrane particles or exosomes derived from said feeder cells.

8. Also disclosed herein are methods of generating TGF-β resistant NK cells of any preceding aspect wherein the feeder cells further comprising at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL.

9. In one aspect, disclosed herein are methods of generating TGF-β resistant NK cells of any preceding aspect, wherein the NK cells comprise memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source (such as for example, an autologous donor, allogeneic donor, or syngeneic donor).

10. Also disclosed herein are methods of generating TGF-β resistant NK cells of any preceding aspect, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

11. In one aspect, disclosed herein are TGF-β resistant NK cells made by the method of any preceding aspect.

12. Also disclosed herein are methods of treating, inhibiting, reducing, decreasing, ameliorating, and/or preventing a cancer and/or metastasis in a subject comprising administering to the subject the TGF-β resistant NK cell of any preceding aspect. For example, disclosed herein are methods of treating, inhibiting, reducing, decreasing, ameliorating, and/or preventing a cancer and/or metastasis (for example, a solid tumor) in a subject with a TGF-β resistant NK cell comprising obtaining an NK cell; culturing the NK cell in the presence of feeder cells (including, but not limited to PBMCs, RPMI8866, HFWT, K562 cells, EBV-LCL, NK cells transfected with membrane bound IL-21, NK cells transfected with membrane bound 4-1BBL, NK cells transfected with membrane bound IL-15 and 4-1BBL , or NK cells transfected with membrane bound IL-21 and 4-1BBL) engineered to express TGF-β thereby creating a TGF-β resistant NK cell or in the presence of plasma membrane particles or exosomes derived from said feeder cells; and administering to the subject the TGF-β resistant NK cell. Also disclosed herein is a TGF-β resistant NK cell of the invention for use as a medicament, preferably for use in a method of treating cancer in a subject. For example, disclosed herein is a TGF-β resistant NK cell for use in a method of treating, inhibiting, reducing, decreasing, ameliorating, and/or preventing a cancer and/or metastasis or a solid tumor in a subject; wherein said TGF-β resistant NK cell is obtainable by a method of generating a TGF-β resistant NK cell of the invention, preferably a method comprising—obtaining an NK cell;—culturing the NK cell in the presence of feeder cells (including, but not limited to PBMCs, RPMI8866, NK-92, NK-92MI, NK-YTS, NK, NKL, ML, ML C.2, NK 3.3, NK-YS, HFWT, K562 cells, EBV-LCL or NK cells) engineered to express TGF-β thereby generating a TGF-β resistant NK cell or culturing the NK cell in the presence of plasma membrane particles or exosomes derived from said feeder cells thereby generating a TGF-β resistant NK cell. Preferably, the feeder cells (including but not limited to PBMCs, RPMI8866, NK-92, NK-92MI, NK-YTS, NK, NKL, ML, ML C.2, NK 3.3, NK-YS, HFWT, K562 cells, EBV-LCL or NK cells) are (i) transfected with membrane bound IL-21, (ii) transfected with membrane bound 4-1BBL, (iii) transfected with membrane bound IL-15 and 4-1BBL or (iv) transfected with membrane bound IL-21 and 4-1BBL. Also disclosed herein is a use of a TGF-β resistant NK cell of the invention for the manufacture of a medicament for the treatment of cancer in a subject. For example, disclosed herein is a use of a TGF-β resistant NK cell for the manufacture of a medicament for treating, inhibiting, reducing, decreasing, ameliorating, and/or preventing a cancer and/or metastasis or a solid tumor in a subject; wherein said TGF-β resistant NK cell is obtainable by a method of generating a TGF-β resistant NK cell of the invention, preferably a method comprising the steps of—obtaining an NK cell;—culturing the NK cell in the presence of feeder cells (including, but not limited to PBMCs, RPMI8866, NK-92, NK-92MI, NK-YTS, NK, NKL, ML, ML C.2, NK 3.3, NK-YS, HFWT, K562 cells, EBV-LCL or NK cells) engineered to express TGF-β thereby generating a TGF-β resistant NK cell or culturing the NK cell in the presence of plasma membrane particles or exosomes derived from said feeder cells thereby generating a TGF-β resistant NK cell. Preferably, the feeder cells (including but not limited to PBMCs, RPMI8866, NK-92, NK-92MI, NK-YTS, NK, NKL, ML, ML C.2, NK 3.3, NK-YS, HFWT, K562 cells, EBV-LCL or NK cells) are (i) transfected with membrane bound IL-21, (ii) transfected with membrane bound 4-1BBL, (iii) transfected with membrane bound IL-15 and 4-1BBL or (iv) transfected with membrane bound IL-21 and 4-1BBL.

13. In one aspect, disclosed herein are methods of treating, inhibiting, reducing, decreasing, ameliorating, and/or preventing a cancer and/or metastasis of any preceding aspect, wherein the NK cells are memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source (such as for example, an autologous donor, allogeneic donor, or syngeneic donor).

14. Also disclosed herein are methods of treating, inhibiting, reducing, decreasing, ameliorating, and/or preventing a cancer and/or metastasis of any preceding aspect, wherein the feeder cells further comprising at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL. Preferably, the TGF-β is membrane bound. Preferably, the at least one additional NK cell effector agent is a membrane bound NK cell effector agent.

15. In one aspect, disclosed herein are methods of treating, inhibiting, reducing, decreasing, ameliorating, and/or preventing a cancer and/or metastasis or a solid tumor of any preceding aspect, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

### III. BRIEF DESCRIPTION OF THE DRAWINGS

16. The accompanying drawings, which are incorporated in and constitute a part of this specification, illustrate several embodiments and together with the description illustrate the disclosed compositions and methods.

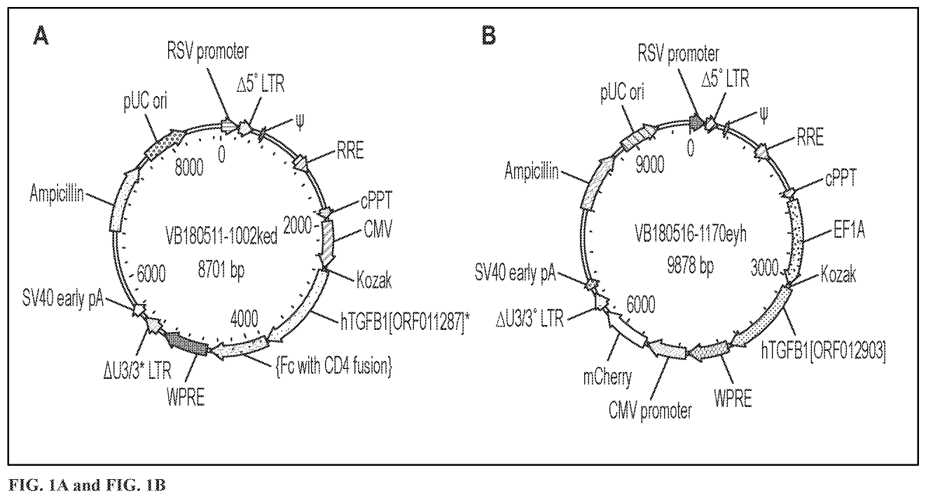

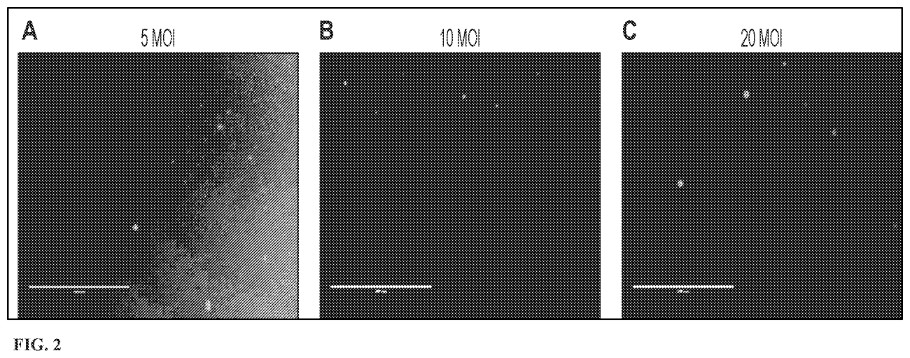

17. FIGS. 1A and 1B show a schematic showing lentiviral vectors expressing the human transforming growth factor beta-1 (hTGFB). FIG. 1A shows membrane bound TGFP (mbTGFb), human TGFP mRNA (1170 bp) was fused to CD4 transmembrane region and IgG4 domain under the control of CMV promoter and cloned into a lentivrial vector. FIG. 1B shows Mcherry-TGFP (mc-TGFB) vector was generated by cloning human TGFP mRNA under control of EF1A promoter and expressing mcherry protein using a CMV promoter.

18. FIG. 2 shows Fluorescent images showing GFP expression in cells. Panels A-C show images of K562 cells, clone (CXT002) infected with lentivirus expressing GFP 48hrs post infection with indicated multiplicity of infection (MOI).

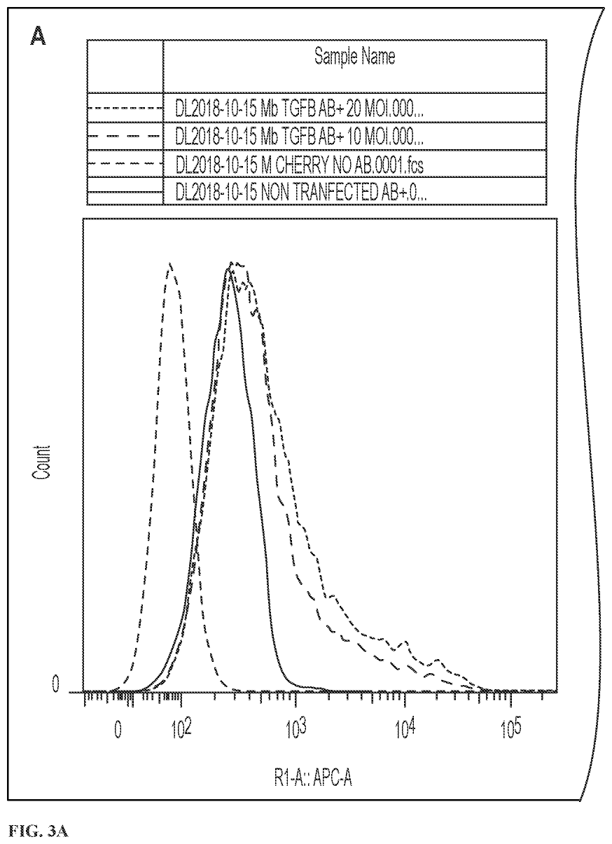

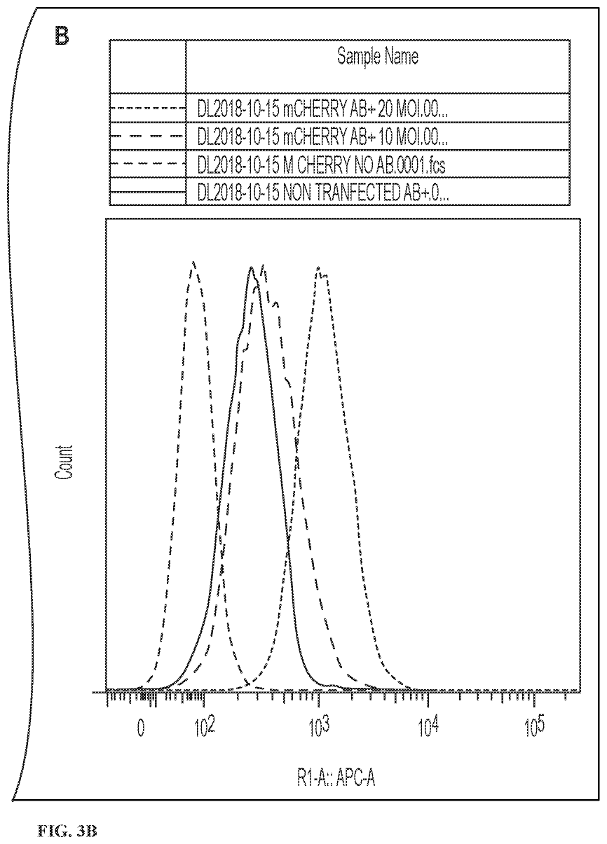

19. FIGS. 3A and 3B show FACS analysis showing TGFP expression. K562 cell clone (CSTX002) was infected with lentivirus expressing membrane bound TGFP (mbTGFβ) or mcherry TGFP (mc-TGFβ) with indicated multiplicity of infection (MOI). The cells were collected 5-days post infection and stained with human anti-TGFB antibody. FIG. 3A shows cells overexpressing membrane bound TGFP (mbTGFβ). FIG. 3B shows cells overexpressing mcherry TGFP (mc-TGFβ).

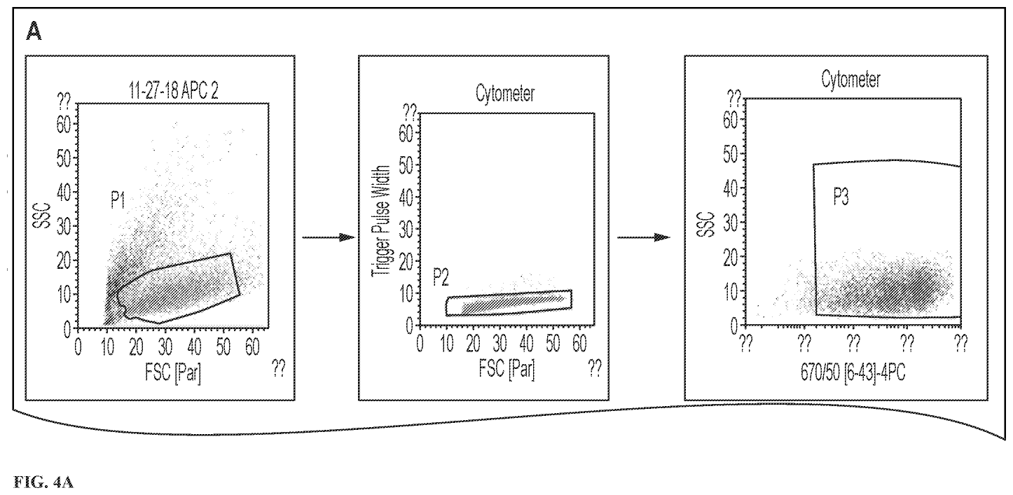

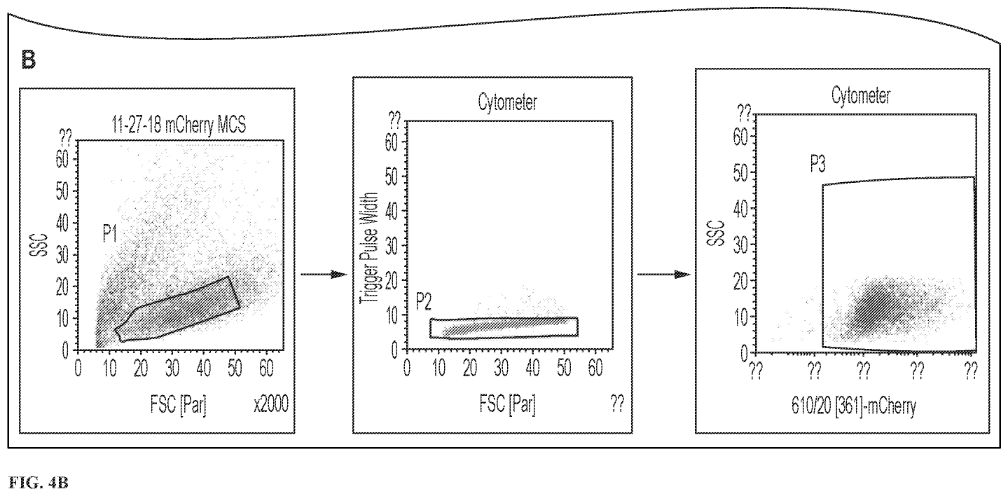

20. FIGS. 4A and 4B show scatter plots showing gating strategy used to sort mbTGFb or mc-TGFB positive cells. Single cells clones were cultured for 10-14 days. FIG. 4A shows mbTGFP cells were stained with anti-TGFP antibody. APC positive cells were collected. FIG. 4B shows mcherry TGFP positive cells were sorted using the fluorescent marker.

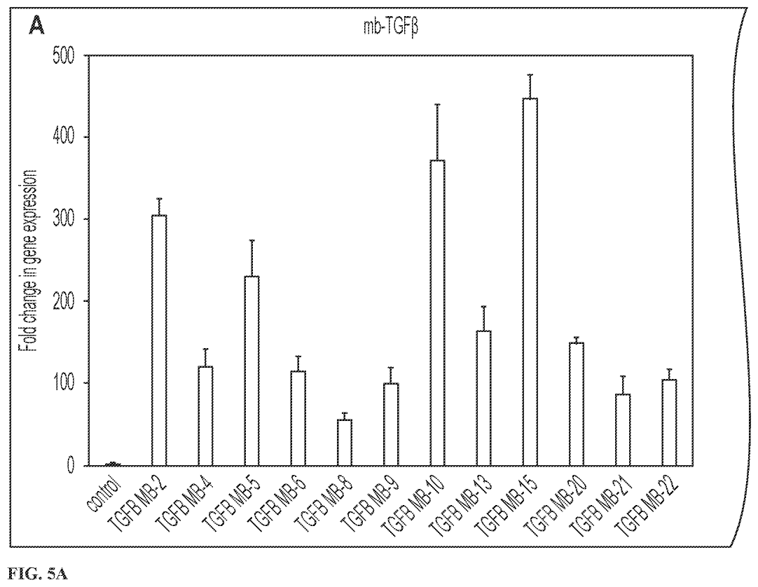

21. FIGS. 5A and 5B show quantitative real time PCR (RT-PCR) showing the expression of TGFβ. RNA was isolated from single cell clone and levels of TGF transcript was determined using RT-PCR. The results are present as fold change relative to the non-transfected control cells. TGF levels in cells infected with membrane bound TGF (mb TGFβ) (5A) or mcherrry TGFP (TGFβ) (5B).

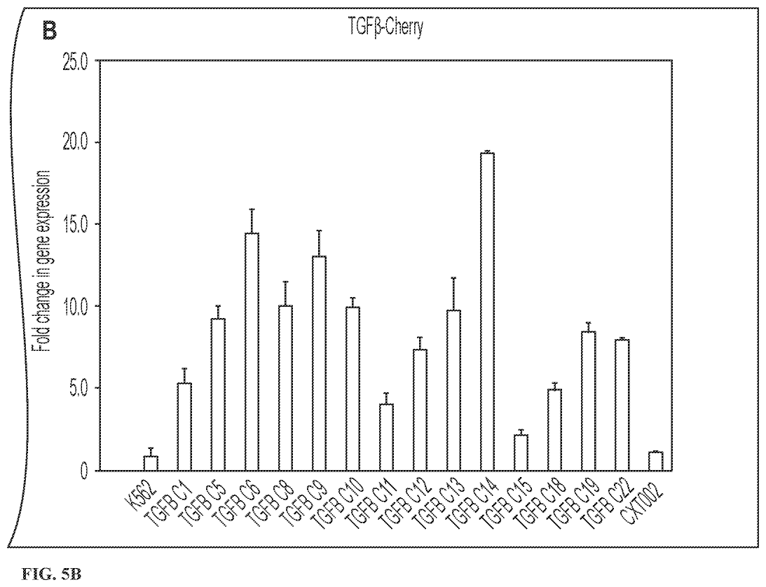

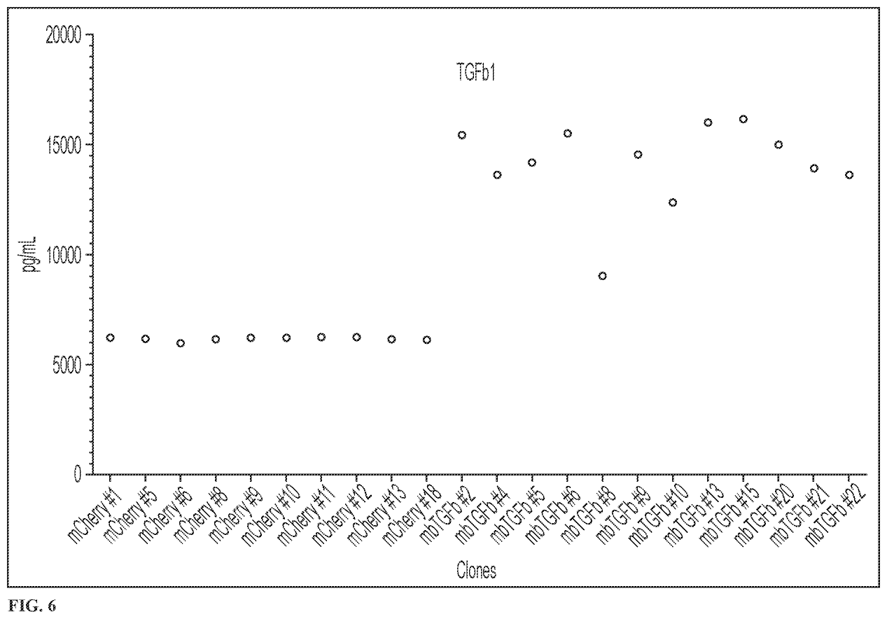

22. FIG. 6 shows an ELISA showing the levels of human TGFP. The single cells clone sorted from mbTGFP and mc-TGFP cells (0.5×10\hat{ }6) were cultured in 96 well plate. The supernatants were collected after 16 hours and the levels of TGFP1 was determined using human TGFβ1 ELISA kit.

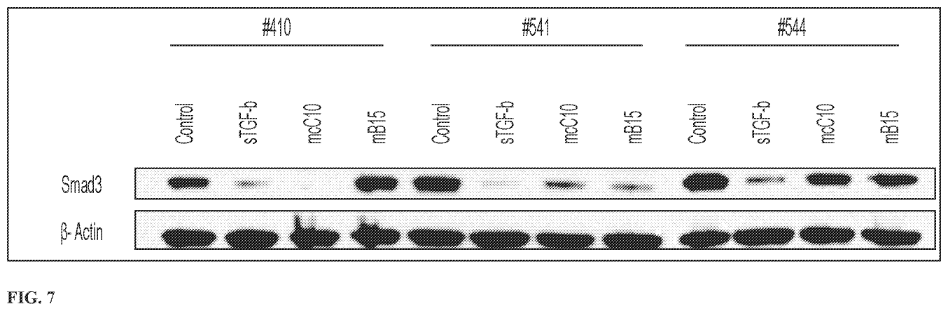

23. FIG. 7 shows an immunoblot showing Smad3 expression in NK cells treated with TGFP. NK cells isolated from normal donor leukopak were expanded with irradiated normal feeder cells CSTX0002 (control), soluble TGFβ (s TGFβ) (long/ml) or with TGFβ overexpressing feeder cell lines (mcC10 or MB15) for a period of two weeks.

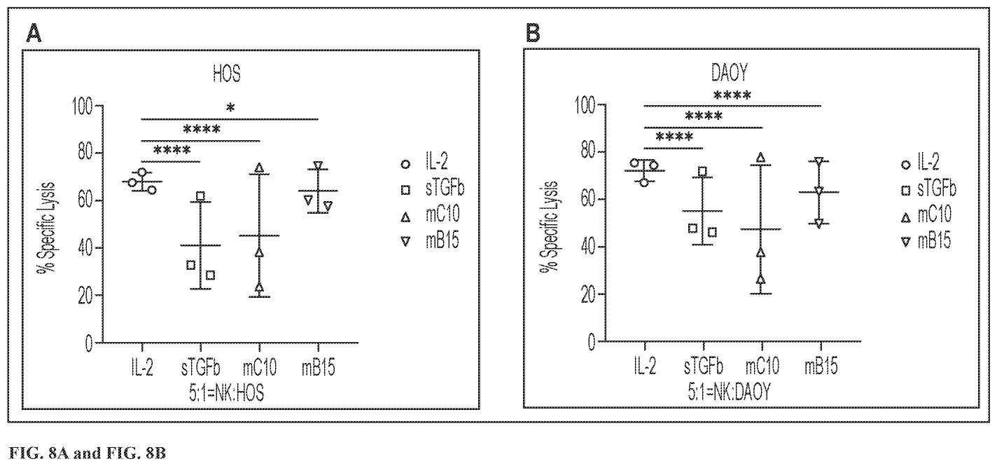

24. FIGS. 8A and 8B show Standard NK cell cytotoxicity assays against tumor cells. NK cells from the peripheral blood of normal donors were co-cultured with calcein labeled tumors at effector to target (E:T) of 5:1 for 4hr. The calcein release in the supernatant was measured using a microplate reader and was used to determine mean specific lysis. NK cells co-cultured with human osteosarcoma cells HOB (8A) Human medulloblastoma cells DAOY (8B). Significance was determined two-way ANOVA with Holm-Sidak's multiple comparisons test. (* p≤0.05, ** p≤0.01, *** p≤0.001, **** p≤0.0001)

### IV. DETAILED DESCRIPTION

25. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

26. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

27. When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

28. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

29. As used herein, the terms “optional” or “optionally” mean that the subsequently described event or circumstance may or may not occur, and that the description includes instances where said event or circumstance occurs and instances where it does not.

30. As used herein, “N-terminal side” or “amino terminal end” refers to directionality of a peptide, polypeptide, or protein and may not mean the N-terminus. In some aspects, where a chimeric or fusion peptide, polypeptide, or protein is discussed, the N-terminal side may refer only to a component of the chimeric or fusion peptide, polypeptide, or protein and not the entire structure. For example, where a Fc domain is discussed, and the Fc domain is described as fused with its amino terminal end or N-terminal side facing intracellularly, contemplated herein are chimeric or fusion peptides, polypeptides, or proteins wherein the signal anchor is at the N-terminus of the chimeric or fusion construct and actually spans the cellular membrane. Thus, in such a chimera, the trans-membrane anchor is attached to the amino terminal side of the Fc domain, with the directionality of the Fc domain has the N-terminal side facing the cell which is inverted relative to an Fc domain on a typical B cell which would typically have the carboxy end spanning the cellular membrane and amino terminal end extending to the extracellular matrix.

31. The terms “peptide,” “polypeptide” and “protein” are used interchangeably to refer to a polymer of amino acid residues.

32. The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine. Preferably, a percentage sequence identity is calculated over the whole length of sequences that are to be compared.

33. Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

34. As various changes could be made in the above-described cells and methods without departing from the scope of the invention, it is intended that all matter contained in the above description and in the examples given below, shall be interpreted as illustrative and not in a limiting sense.

35. An “increase” can refer to any change that results in a greater amount of a symptom, disease, composition, condition or activity. An increase can be any individual, median, or average increase in a condition, symptom, activity, composition in a statistically significant amount relative to a control. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

36. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount relative to a control. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

37. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

38. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

39. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

40. The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

41. The term “therapeutically effective” refers to the amount of the composition used is of sufficient quantity to ameliorate one or more causes or symptoms of a disease or disorder. Such amelioration only requires a reduction or alteration, not necessarily elimination.

42. The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

43. “Administration” to a subject includes any route of introducing or delivering to a subject an agent. Administration can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

44. “Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

45. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

46. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TGF-β expressing engineered feeder cell is disclosed and discussed and a number of modifications that can be made to a number of molecules including the TGF-β expressing engineered feeder cell are discussed, specifically contemplated is each and every combination and permutation of TGF-β expressing engineered feeder cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

47. The use of NK cells in anti-tumor therapy is becoming increasing utilized. However, studies have shown that cancers secrete TGF-β in defense of the killing action of NK cells. The secretion of TGF-β results in decreased killing. Previous efforts to address the decreased killing have included the use of TGF-β inhibitors; however, the use of TGF-β inhibitors can cause adverse side effects systemically. Alternatively, clinical grade TGF-β could be used during the expansion of NK cells but, the use of clinical grade TGF-β is both laborious and prohibitively expensive due to the excessive large quantities of an already expensive reagent (i.e., TGF-β) that are needed to observe any effect. To address the need for TGF-β resistant NK cells and avoid shortfalls of either TGF-β inhibition or soluble TGF-β culture supplements, herein disclosed are engineered feeder cells that have been modified to express soluble or membrane bound TGF-β.

48. Also disclosed herein are engineered feeder cells, further comprising at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL. Preferably, the at least one additional NK cell effector agent is a membrane bound NK cell effector agent.

49. It is understood and herein contemplated that the feeder cells can be derived from any source for feeder cells that can expand and/or activate NK cells. For example, the feeder cells comprise PBMCs, RPMI8866, HFWT, K562 cells, EBV-LCL, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-21, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound 4-1BBL, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-15 and 4-1BBL, or NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-21 and 4-1BBL.

50. Additionally, also disclosed herein are plasma membrane particles or exosomes derived from any of the engineered feeder cells disclosed herein or made by the methods disclosed herein.

1. Delivery of the Compositions to Cells

51. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

52. utilizing endonuclease ribonucleoprotein complexes (such as, for example, Cas9/RNPs) to reprogram (i.e., engineer or modify) feeder cells.

53. Endonuclease/RNPs (for example, a Cas9/RNP) are comprised of three components, recombinant endonuclease protein (for example, a Cas9 endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of a RNA that can hybridize to a target sequence complexed complementary repeat RNA (crRNA) and trans complementary repeat RNA (tracrRNA). Accordingly the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. These Cas9/RNPs are capable of cleaving genomic targets with higher efficiency as compared to foreign DNA-dependent approaches due to their delivery as functional complexes. Additionally, rapid clearance of Cas9/RNPs from the cells can reduce the off-target effects such as induction of apoptosis. Accordingly, in one aspect, disclosed here are methods of genetically modifying an NK cell comprising obtaining guide RNA (gRNA) specific for a target DNA sequence in the NK cell; and b) transducing (for example, introducing via electroporation) into a target NK cell, a ribonucleoprotein (RNP) complex comprising a class 2 CRISPR/Cas endonuclease (Cas9) complexed with a corresponding CRISPR/Cas guide RNA that hybridizes to the target sequence within the genomic DNA of the feeder cell.

### a) Nucleic Acid Based Delivery Systems

54. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

55. As used herein, plasmid or viral vectors are agents that transport the disclosed nucleic acids, such as TGF-β, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

56. Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

57. A retrovirus is an animal virus belonging to the virus family of Retroviridae, including any types, subfamilies, genus, or tropisms.

58. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle.

This amount of nucleic acid is sufficient for the delivery of a one to many genes depending on the size of each transcript. It is preferable to include either positive or negative selectable markers along with other genes in the insert.

59. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. In one aspect, the nucleic acid encoding the TGF-b can be delivered via a lentiviral vector

### (2) Adenoviral Vectors

60. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

61. A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

62. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. AAVs used can be derived from any AAV serotype, including but not limited to AAC1, AAV2, AAV3, AAV4, AAV5, AAV6, AAV7, AAV8, AAV9, and recombinant (rAAV) such as, for example AAV-Rh74, and/or synthetic AAV (such as, for example AAV-DJ, Anc80). AAV serotypes can be selected based on cell or tissue tropism. AAV vectors for use in the disclosed compositions and methods can be single stranded (SS) or self-complementary (SC).

63. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

64. Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression.

65. The disclosed vectors thus provide DNA molecules which are capable of integration into a mammalian chromosome without substantial toxicity.

66. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

67. It is understood and herein contemplated that the packaging capacity of an AAV is limited. One method to overcome the loading capacity of an AAV vector is through the use of 2 vectors, wherein the transgene is split between the two plasmids and a 3′ splice donor and 5′ splice acceptor are used to join the two sections of transgene into a single full-length transgene. Alternatively, the two transgenes can be made with substantial overlap and homologous recombination will join the two segments into a full-length transcript.

2. Expression Systems

68. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements. Promoters used to make the disclosed feeder cells expressing TGF-β (either soluble or membrane bound) can be constitutive or inducible.

### a) Viral Promoters and Enhancers

69. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein. In one aspect, the TGF-b mRNA can be expressed under control of an EF1A promoter or a CMV promoter.

70. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

71. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

72. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

73. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

74. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

75. The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

76. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are:

CHO DHFR- cells and mouse LTK- cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

77. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R.C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 3. Pharmaceutical Carriers/Delivery of Pharmaceutical Products

78. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

79. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

80. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

81. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Banelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

82. The compositions, including antibodies, can be used therapeutically in combination with a pharmaceutically acceptable carrier.

83. Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

84. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

85. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

86. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

87. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

88. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

89. Compositions for oral administration include powders or granules, suspensions or solutions in water or non-aqueous media, capsules, sachets, or tablets. Thickeners, flavorings, diluents, emulsifiers, dispersing aids or binders may be desirable.

90. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

91. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHODS OF MAKING TGF-β RESISTANT NK CELLS

92. As noted throughout the primary purpose of the disclosed feeder cells is to make NK cells that are resistant to TGF-β. Accordingly, in one aspect, disclosed herein are methods of generating TGF-β resistant NK cells, comprising incubating NK cells in the presence of the engineered feeder cells, plasma membrane particles, or exosome disclosed herein. For example, disclosed herein are methods of generating TGF-β resistant NK cells, comprising incubating NK cells in the presence of feeder cells (including but not limited to PBMCs, RPMI8866, HFWT, K562 cells, EBV-LCL, NK cells (including, but not limited to NK-92, NK-92MI, NK-YTS, NK, NKL, ML, ML C.2, NK 3.3, NK-YS) transfected with membrane bound IL-21, NK cells (including, but not limited to NK-92, NK-92MI, NK-YTS, NK, NKL, ML, ML C.2, NK 3.3, NK-YS) transfected with membrane bound 4-1BBL, NK cells (including, but not limited to NK-92, NK-92MI, NK-YTS, NK, NKL, ML, ML C.2, NK 3.3, NK-YS) transfected with membrane bound IL-15 and 4-1BBL , or NK cells (including, but not limited to NK-92, NK-92MI, NK-YTS, NK, NKL, ML, ML C.2, NK 3.3, NK-YS) transfected with membrane bound IL-21 and 4-1BBL) that have been engineered to express TGF-β or incubating NK cells in the presence of plasma membrane particles or exosomes derived from said feeder cells. Preferably, the TGF-β expressed by the feeder cells is membrane bound.

93. In one aspect, disclosed herein are methods of generating TGF-β resistant NK cells wherein the feeder cells comprise at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL. Preferably, the at least one additional NK cell effector agent is a membrane bound NK cell effector agent.

94. It is understood and herein contemplated that the methods of generating TGF-β resistant NK cells disclosed herein can be used on any NK cell (exogenous or endogenous) where resistance TGF-β is desired. Accordingly, disclosed herein are methods of generating TGF-β resistant NK cells wherein the NK cells comprise memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source (such as for example, an autologous donor, allogeneic donor, or syngeneic donor).

95. To generate TGF-β resistant NK cells the NK cells must be exposed to the feeder cell, exosome, or plasma membrane particle expressing TGF-β (on its membrane or soluble) for a period of time to confer resistance. Thus, in one aspect, disclosed herein are methods of generating TGF-β resistant NK cells, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days. It is further understood and herein contemplated that the NK cells can be cultured for additional periods of time after exposure to the engineered feeder cells or plasma membrane particles or exosomes derived from said feeder cells. In one aspect, the NK cells can be contacted with the engineered feeder cells or plasma membrane particles or exosomes derived from said feeder cells for between 7 and 21 days, preferably between 7 and 14 days.

96. As noted throughout, the disclosed methods generate NK cells that are TGF-β resistant Thus, in one aspect, disclosed herein are TGF-β resistant NK cells made by the method of generating TGF-β resistant NK cells disclosed herein.

97. In some instances, the plasma membrane particles or exosome derived from the engineered feeder cells can be obtained by nitrogen cavitation.

98. In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

### D. METHOD OF TREATING CANCER

99. The disclosed compositions can be used to treat any disease where uncontrolled cellular proliferation occurs such as cancers. Accordingly, disclosed herein are methods of treating, inhibiting, reducing, decreasing, ameliorating, and/or preventing a cancer and/or metastasis in a subject comprising administering to the subject the TGF-β resistant NK cell disclosed herein. For example, disclosed herein are methods of treating, inhibiting, reducing, decreasing, ameliorating, and/or preventing a cancer and/or metastasis (such as, for example, a solid tumor) in a subject with a TGF-β resistant NK cell comprising obtaining an NK cell; culturing the cell in the presence of feeder cells (including, but not limited to PBMCs, RPMI8866, HFWT, K562 cells, EBV-LCL, NK cells (including, but not limited to NK-92, NK-92MI, NK-YTS, NK, NKL, ML, ML C.2, NK 3.3, NK-YS) transfected with membrane bound IL-21, NK cells (including, but not limited to NK-92, NK-92MI, NK-YTS, NK, NKL, ML, ML C.2, NK 3.3, NK-YS) transfected with membrane bound 4-1BBL, NK cells (including, but not limited to NK-92, NK-92MI, NK-YTS, NK, NKL, ML, ML C.2, NK 3.3, NK-YS) transfected with membrane bound IL-15 and 4-1BBL , or NK cells (including, but not limited to NK-92, NK-92MI, NK-YTS, NK, NKL, ML, ML C.2, NK 3.3, NK-YS transfected with membrane bound IL-21 and 4-1BBL) engineered to express TGF-β thereby creating a TGF-β resistant NK cell or in the presence of plasma membrane particles or exosomes derived from said feeder cells; and administering to the subject the TGF-β resistant NK cell. Preferably, the TGF-β expressed by the feeder cell is membrane bound.

100. It is understood and herein contemplated that feeder cells can be used in the disclosed methods of treatment not only to make TGF-β resistant and also activate and/or expand exogenous NK cell populations (such as, for example, memory-like NK cells such as NKG2C⁺, CD56^{bright} NK cells, CD56^{dim} NK cells, peripheral NK cells, NK T cells, or tumor infiltrating NK cells), but can be used to make endogenous NK cell populations TGF-β resistant and/or activate and/or expand endogenous NK cell populations (such as, for example, memory-like NK cells such as NKG2C⁺, CD56^{bright} NK cells, CD56^{dim} NK cells, peripheral NK cells, NK T cells, or tumor infiltrating NK cells). The NK cells which can be used in the methods of treating a cancer can comprise any NK cell from a donor or NK cell line. In one aspect, disclosed herein are methods of treating, inhibiting, reducing, decreasing, ameliorating, and/or preventing a cancer and/or metastasis, wherein the NK cells are memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source (such as for example, an autologous donor, allogeneic donor, or syngeneic donor). In one aspect, the NK cell can be an endogenous NK cell that is made resistant in vivo through exposure to the engineered feeder cells and/or plasma membrane particles or exosomes derived from said feeder cells disclosed herein.

101. The feeder cells, plasma membrane particles, and/or exosomes used in the disclosed methods of treatment can further comprise additional effector agents to expand and/or activate NK cells. Thus, in one aspect disclosed herein are methods of treating, inhibiting, reducing, decreasing, ameliorating, and/or preventing a cancer and/or metastasis, wherein the feeder cells further comprising at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL. Preferably, said cytokine, adhesion molecule or NK cell activating agent is a human cytokine, adhesion molecule or NK cell activating agent. Preferably, the at least one additional NK cell effector agent is a membrane bound NK cell effector agent.

102. In one aspect, disclosed herein are methods of treating, inhibiting, reducing, decreasing, ameliorating, and/or preventing a cancer and/or metastasis, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

103. The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

### E. EXAMPLES

104. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Generate Genetically-Modified Feeder Cell Clones Consisting of K562 Expressing mbIL21, CD137L, and Secreted or mbTGFβ

105. Multiple feeder cell lines overexpressing human TGFβ1 were generated by transducing the K562-based feeder cell clone CSTX002 with lentiviral vectors expressing TGFβ1 as a membrane-bound mutein (mbTGFβ) or in native secreted form. The CSTX002 clone expresses a membrane-bound mutein of the cytokine IL-21 and the co-stimulatory molecule 4-1BBL (CD137). Briefly, the human mbTGFβ1 fusion construct was generated in silico using human TGFβ1 fused to CD4 transmembrane region and IgG4 (F_{C}) stalk under the regulatory control of the human CMV promoter. The construct then underwent codon optimization and validation of sequence homologies. Similarly, the bicistronic mc-TGFβ vector was generated using human TGFβ is under the control of human elongation factor-1 (EF1A) promoter with independent expression of the fluorescent protein mcherry under the CMV promoter. Detailed vector maps showing the design of the two constructs (mbTGFβ and mc-TGFβ) are shown in FIG. 1. Both constructs were cloned into a 3^{rd} generation lentiviral system that was used to generate high-titer lentivirus.

106. Since lentiviral transduction efficiency can vary amongst cell lines and the initial transduction of CSTX002 was with lentivirus encoding green fluorescent protein (GFP), susceptibility to superinfection was verified at three different viral titers (multiplicity of infection (MOI) of 5, 10, 20) using a control lentivirus encoding GFP (FIG. 2). Good GFP expression was observed in each condition at 48 hours post transfection. Infection of CSTX002 was preceded at MOI 5, 10 and 20 of the TGFβ vectors, and stained the transduced cells with human TGFβ1 antibody (Biolegend Cat # 349615) 5-days post infection (FIG. 3). Optimal expression of TGFβ was observed at 20 MOI for cells infected with either of the vectors (mbTGFβ or mc-TGFβ) so all subsequent experiments were performed using 20 MOI. To generate clones overexpressing mbTGFβ or mc-TGFP, CSTX002 were infected with the two viruses as described above. Following the infection, the cells were cultured for 4-5 days. The mbTGFβ cells were stained with TGFβ1 antibody and sorted on BD Influx using the BD software program. The positive cells were sorted directly into 96-well plates. The mc-TGFP positive cells were sorted using the using green laser (561nm) to collect mcherry positive cells. Single-cell clones were collected from 96-well plate and the expression of mbTGFβ or mc-TGFβ was determined using FACS (FIG. 4). In addition, mRNA expression was determined using quantitative real time PCR (RT-PCR) using hTGFβ1 TaqMan primers. There was an increase in TGFβ mRNA expression by 5-20 fold in mc-TGFP cells and 50-400 fold increase in mbTGFβ cells compared to the non-transfected cells controls (FIG. 5).

### 2. Example 2

### Demonstrate that Propagation of NK Cells on these Novel Feeder Cells Results in TGFβ Resistant NK Cells, and describe the Characteristics of these Resistant Cells

107. It was then determined whether the new feeder cells transduced with mbTGFβ and mc-TGFβ secrete intact TGFβ. mbTGFβ and mc-TGFβ cells were seeded in a 96 well plate following a 16 hour incubation the supernatants were collected and stored at −80C. The level of TGFβ1 was determined using the TGFβ1 ELISA kit (R&D Cat DB100B). All clones from both expression vectors were found to secrete TGFβ. The levels were 5000 pg/ml for mc-TGFβ cells, and ranged 10,000-15,000 pg/ml for mbTGFβ cells (FIG. 6).

108. TGFβ is an immune suppressive molecule that attenuate the anti-tumor function of NK cells through activation of downstream targets Smad2/Smad3. The addition of soluble

TGFβ1 during expansion of NK cells with CSTX002 results in a marked reduction in Smad3 expression in NK cells which renders them cytokine hyper-secreting and less susceptible to TGFβ1 signaling. To determine whether the newly-transduced clones could induce a similar remodeling off NK cells, NK cells from 3 healthy donors were expanded using normal CSTX002, CSTX002 with soluble TGFβ1, or with the new TGFβ1-expressing CSTX002.

Briefly, the feeder cells were irradiated at 100 Gray (Gy) , and NK cells isolated from PBMCs of healthy blood donors were expanded using the irradiated feeder cell for two weeks in presence of low dose IL-2 (50 IU/ml). Similar reduction in levels of Smad3 expression in NK cells expanded with mbTGFβ and mc-TGFβ feeder cells (FIG. 7) were observed.

109. It was then determined whether the NK cells expanded with mbTGFβ and mc-TGFβ transduced feeder cells were functionally similar to those cultured with soluble TGFβ and CSTX002. Following the expansion, NK cells were co-cultured with calcein-loaded HOS (osteosarcoma) or DAOY (medulloblastoma) target cells at 5:1 for 4 hours. The calcein release was used to determine mean percent cell lysis. NK treated with 10 ng/ml of soluble human TGFβ1 (sTGFβ) served as a positive control. As shown in FIG. 8, NK cells grown with feeder cells mbTGFβ, mc-TGFβ or expanded in presence of soluble TGFβ had significantly reduce ability to lyse target tumor cells HOS and DAOY compared to NK cells expanded with control feeder cells. This is consistent with TGFβ-imprinted NK cells.

What is claimed is:

1. An engineered feeder cell that has been modified to express soluble or membrane bound TGF-β.

2. The engineered feeder cell of claim 1, wherein TGF-β is expressed by an inducible or constitutive promoter.

3. The engineered feeder cell of claim 1 or 2, wherein the feeder cell comprises a leukemia K562 cell.

4. The engineered feeder cell of claim 1 or 2, wherein the feeder cell comprises a NK cell.

5. The engineered feeder cell of any of claims 1-3, further comprising at least one additional NK cell effector agent on its cell surface, wherein the at least one additional NK cell effector agent is a cytokine, an adhesion molecule, or an NK cell activating agent.

6. The engineered feeder cell of claim 5, wherein the at least one additional NK cell effector agent is selected from 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists.

7. The engineered feeder cell of claim 5, wherein the at least one additional NK cell effector agent comprises IL-21, 4-1BBL, IL-15, IL-21 and 4-1BBL, IL-21 and IL-15, or IL-15 and 4-1BBL.

8. The engineered feeder cell of any of claims 1-7, wherein the feeder cells comprise PBMCs, RPMI8866, HFWT, K562 cells, EBV-LCL, NK cells transfected with membrane bound IL-21, NK cells transfected with membrane bound 4-1BBL, NK cells transfected with membrane bound IL-15 and 4-1BBL , or NK cells transfected with membrane bound IL-21 and 4-1BBL.

9. A plasma membrane particle or exosome derived from the engineered feeder cell of any of claims 1-8.

10. A method of generating TGF-β resistant NK cells, comprising incubating NK cells in the presence of the engineered feeder cells of any of claim 1-8 or the exosome or plasma membrane particle of claim 9.

11. A method of generating TGF-β resistant NK cells, comprising incubating NK cells in the presence of feeder cells that have been engineered to express TGF-β or incubating NK cells in the presence of plasma membrane particles or exosomes derived from said feeder cells.

12. The method of generating TGF-β resistant NK cells of claim 11, wherein the feeder cells comprise PBMCs, RPMI8866, HFWT, K562 cells, EBV-LCL, NK cells transfected with membrane bound IL-21, NK cells transfected with membrane bound 4-1BBL, NK cells transfected with membrane bound IL-15 and 4-1BBL , or NK cells transfected with membrane bound IL-21 and 4-1BBL.

13. The method of generating TGF-β resistant NK cells of claim 11 or 12, wherein the feeder cells, further comprising at least one additional NK cell effector agent on its cell surface, wherein the at least one additional NK cell effector agent is a cytokine, an adhesion molecule, or an NK cell activating agent.

14. The method of generating TGF-β resistant NK cells of claim 13, wherein the at least one additional NK cell effector agent is selected from 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists.

15. The method of generating TGF-β resistant NK cells of claim 14, wherein the at least one additional NK cell effector agent comprises IL-21, 4-1BBL, IL-15, IL-21 and 4-1BBL, IL-21 and IL-15, or IL-15 and 4-1BBL.

16. The method of generating TGF-β resistant NK cells of any of claims 11-15, wherein the NK cells comprise memory-like NK cells such as NKG2C⁺, CD56^{bright} NK cells, CD56^{dim} NK cells, peripheral NK cells, and NK T cells, tumor infiltrating NK cells.

17. The method of generating TGF-β resistant NK cells of any of claims 11-16, wherein the NK cells are obtained from a donor subject.

18. The method of generating TGF-β resistant NK cells of any of claims 11-16, wherein the NK cells are obtained from autologous donor, allogeneic donor, or syngeneic donor.

19. The method of generating TGF-β resistant NK cells of any of claims 11-18, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

20. A TGF-β resistant NK cell made by the method of any of claims 10-19.

21. A method of treating a cancer in a subject comprising administering to the subject the TGF-β resistant NK cell of claim 20.

22. A method of treating a cancer in a subject with a TGF-β resistant NK cell comprising obtaining an NK cell; culturing the cell in the presence of feeder cells engineered to express TGF-β thereby creating a TGF-β resistant NK cell or in the presence of of plasma membrane particles or exosomes derived from said feeder cells; and administering to the subject the TGF-β resistant NK cell.

23. The method of treating a cancer of claim 22, wherein the NK cells are memory-like NK cells such as NKG2C⁺, CD56^{bright} NK cells, CD56^{dim} NK cells, peripheral NK cells, and NK T cells, tumor infiltrating NK cells.

24. The method of treating a cancer of claim 22 or 23, wherein the NK cells are obtained from a donor subject.

25. The method of treating a cancer of any of claims 22-24, wherein the NK cells are obtained from autologous donor, allogeneic donor, or syngeneic donor.

26. The method of treating a cancer of any of claims 22-25, wherein the wherein the feeder cells comprise PBMCs, RPMI8866, HFWT, K562 cells, EBV-LCL, NK cells transfected with membrane bound IL-21, NK cells transfected with membrane bound 4-1BBL, NK cells transfected with membrane bound IL-15 and 4-1BBL , or NK cells transfected with membrane bound IL-21 and 4-1BBL.

27. The method of treating a cancer of any of claims 22-26, wherein the feeder cells further comprise at least one additional NK cell effector agent on its cell surface, wherein the at least one additional NK cell effector agent is a cytokine, an adhesion molecule, or an NK cell activating agent.

28. The method of treating a cancer of claim 27, wherein the at least one additional NK cell effector agent is selected from 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists.

29. The method of treating a solid tumor of claim of claim 28, wherein the at least one additional NK cell effector agent comprises IL-21, 4-1BBL, IL-15, IL-21 and 4-1BBL, IL-21 and IL-15, or IL-15 and 4-1BBL.

30. The method of treating a cancer of any of claims 22-29, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.
